(19) United States
(12) Patent Application Publication
(10) Pub. No.: US 2022/0240795 A1
(43) Pub. Date: Aug. 4, 2022

(54) SPHYGMOMANOMETER, BLOOD PRESSURE MEASUREMENT METHOD, AND COMPUTER-READABLE RECORDING MEDIUM

(71) Applicant: OMRON HEALTHCARE CO., LTD., Muko-shi (JP)

(72) Inventors: Shingo YAMASHITA, Kyoto (JP); Yukiya SAWANOI, Kyoto (JP); Mika EZOE, Kyoto (JP)

(73) Assignee: OMRON HEALTHCARE CO., LTD., Muko-shi (JP)

(21) Appl. No.: 17/724,217

(22) Filed: Apr. 19, 2022

Related U.S. Application Data
(63) Continuation of application No. PCT/JP2020/037772, filed on Oct. 5, 2020.

(30) Foreign Application Priority Data
Oct. 29, 2019 (JP) 2019-196142

(51) Int. Cl. A61B 5/0225; A61B 5/00

(52) U.S. Cl. CPC A61B 5/0225; A61B 5/6824; A61B 2560/0475; A61B 2560/029; A61B 5/7267

(57) ABSTRACT

A sphygmomanometer includes a storage unit that stores a measured blood pressure value. In a nighttime blood pressure measurement mode, blood pressure measurement is automatically started according to a schedule, and a blood pressure is measured when a blood pressure measuring cuff is in a pressurization process or a depressurization process. Further, it is determined whether or not a current blood pressure value measured is different from a past blood pressure value stored in the storage unit beyond an allowable range. When the current blood pressure value is different from the past blood pressure value beyond the allowable range, it is discriminated which phenomenon among a plurality of predetermined types of phenomena has occurred in the subject. A time of remeasurement with respect to a measurement time of the current blood pressure value is variably set according to which phenomenon among the plurality of types of phenomena has occurred.

### CROSS-REFERENCE TO RELATED APPLICATION

This application is based on an application No. 2019-196142 filed in Japan on Oct. 29, 2019, the entire content of which is hereby incorporated by reference.

### TECHNICAL FIELD

The present invention relates to a sphygmomanometer, and more particularly to a sphygmomanometer having a nighttime (sleep) blood pressure measurement mode. Further, the present invention also relates to a blood pressure measurement method of measuring a blood pressure by such a sphygmomanometer. Furthermore, the present invention also relates to a computer-readable recording medium storing a program for causing a computer to execute such a blood pressure measurement method.

### BACKGROUND ART

Conventionally, as this type of sphygmomanometer, for example, Patent Document 1 (WO 2018/168797 A) discloses a sphygmomanometer that measures a blood pressure again after a preset setting time elapses when it is determined that there is a possibility that an error is included in a measured blood pressure value (a posture is bad and a measurement error occurs) in a nighttime (sleep) blood pressure measurement mode.

### SUMMARY OF INVENTION

When nighttime blood pressure measurement is performed for a long time (typically, overnight), a subject can experience various phenomena that can affect a blood pressure value, such as a change in a sleep state, occurrence of an irregular pulse wave, a change in a posture, and body motion.

However, in the conventional sphygmomanometer, when there is a possibility that the current blood pressure value includes a measurement error in the nighttime blood pressure measurement mode, a blood pressure is measured again after a certain setting time elapses. For this reason, when the setting time is too long in spite of the phenomenon that has occurred in the subject (for example, waiting for 30 minutes or longer for body motion for several tens of seconds), there is a problem that a blood pressure is measured again at a time unnecessarily greatly deviated from the time at which the blood pressure measurement should originally be performed, and a blood pressure value at an appropriate time cannot be obtained. On the other hand, if the setting time is too short for the phenomenon that has occurred in the subject, there is a problem that the phenomenon that has occurred is still continuing at the time of remeasurement, and there is a high possibility that a correct blood pressure value cannot be obtained.

Therefore, an object of the present invention is to provide a sphygmomanometer and a blood pressure measurement method capable of appropriately setting the time of remeasurement according to a phenomenon that has occurred in a subject when the current blood pressure value measured in the nighttime blood pressure measurement mode may include a measurement error. Another object of the present invention is to provide a computer-readable recording medium storing a program for causing a computer to execute such a blood pressure measurement method.

In order to achieve the object, a sphygmomanometer of the present disclosure is a sphygmomanometer that performs blood pressure measurement by temporarily pressing a measurement target site of a subject with a blood pressure measuring cuff,

the sphygmomanometer having a nighttime blood pressure measurement mode in which the blood pressure measurement automatically starts according to a predetermined schedule, the sphygmomanometer comprising:

a storage unit that stores a measured blood pressure value;

a blood pressure measurement unit that automatically starts blood pressure measurement according to the schedule in the nighttime blood pressure measurement mode, and measures a blood pressure when the blood pressure measuring cuff is in a pressurization process or a depressurization process;

a difference determination unit that determines whether or not a current blood pressure value measured is different from a past blood pressure value stored in the storage unit beyond a predetermined allowable range;

a phenomenon discrimination unit that discriminates which phenomenon among a plurality of predetermined types of phenomena that can affect a blood pressure value has occurred in the subject when the current blood pressure value is different from the past blood pressure value beyond the allowable range; and

a schedule resetting unit that variably sets a time of remeasurement with respect to a measurement time of the current blood pressure value according to which phenomenon among the plurality of types of phenomena has occurred.

In the present specification, the current blood pressure value is “different from the past blood pressure value beyond a predetermined allowable range” typically means that the current blood pressure value is substantially different from the past blood pressure value in consideration of a measurement error. The “past blood pressure value” may be, for example, a previous blood pressure value obtained according to the schedule in the nighttime blood pressure measurement mode, or may be an average value of nighttime blood pressure values of the previous day obtained according to the schedule in the nighttime blood pressure measurement mode.

The “plurality of predetermined types of phenomena” typically refers to phenomena that can affect a blood pressure value, such as a change in a sleep state, occurrence of an irregular pulse wave, a change in a posture, and body motion. The “change in a sleep state” refers to a change in sleep depth, for example, a change from non-REM sleep (deep sleep) to REM sleep (light sleep), a change from REM sleep (light sleep) to an awake state, or the like. The “occurrence of an irregular pulse wave” refers to a state in which disturbance (including arrhythmia) occurs in a pulse wave that should be originally repeated at a constant cycle and constant intensity. The “change in a posture” refers to a phenomenon in which the subject shifts from a certain posture (for nighttime blood pressure measurements, typically a supine position) to another posture. The “body motion” refers to body motion (for example, repetitive motion) that does not correspond to a change in a posture.

“Whether or not a phenomenon has occurred” means whether or not a phenomenon has occurred at the measurement time of the blood pressure value. Note that a case where the plurality of predetermined types of phenomena have not occurred at all is also subject to the discrimination.

The “measurement time” of the blood pressure value refers to a time when the blood pressure measurement (usually required for about 1 minute to 2 minutes) is automatically started according to the schedule, and has the same meaning as a time when the blood pressure value is actually calculated in the pressurization process or the depressurization process of the blood pressure measuring cuff.

In another aspect, a blood pressure measurement method according to the present disclosure is a blood pressure measurement method for a sphygmomanometer that performs blood pressure measurement by temporarily pressing a measurement target site of a subject with a blood pressure measuring cuff, the sphygmomanometer having a nighttime blood pressure measurement mode in which the blood pressure measurement is automatically started according to a predetermined schedule, and including a storage unit that stores a measured blood pressure value, the blood pressure measurement method comprising:

automatically starting blood pressure measurement according to the schedule in the nighttime blood pressure measurement mode, and measuring a blood pressure when the blood pressure measuring cuff is in a pressurization process or a depressurization process;

determining whether or not a current blood pressure value measured is different from a past blood pressure value stored in the storage unit beyond a predetermined allowable range;

discriminating which phenomenon among a plurality of predetermined types of phenomena that can affect a blood pressure value has occurred in the subject when the current blood pressure value is different from the past blood pressure value beyond the allowable range; and

variably setting a time of remeasurement with respect to a measurement time of the current blood pressure value according to which phenomenon among the plurality of types of phenomena has occurred.

In yet another aspect, a computer-readable recording medium according to the present disclosure is a computer-readable recording medium non-transitorily storing a program for causing a computer to execute the above blood pressure measurement method.

### BRIEF DESCRIPTION OF DRAWINGS

The present invention will become more fully understood from the detailed description given hereinbelow and the accompanying drawings which are given by way of illustration only, and thus are not limitative of the present invention, and wherein:

FIG. 1 is a diagram illustrating an appearance of a wrist-type sphygmomanometer according to an embodiment of the present invention.

FIG. 2 is a diagram illustrating a block configuration of the sphygmomanometer.

FIG. 3 is a diagram illustrating a mode in which the sphygmomanometer is attached to a left wrist as a measurement target site.

FIG. 4A is a diagram illustrating a sitting position as a measurement posture.

FIG. 4B is a view illustrating a supine position as a measurement posture.

FIG. 5 is a diagram illustrating an operation flow when blood pressure measurement is performed in a normal blood pressure measurement mode by the sphygmomanometer.

FIG. 6 is a diagram illustrating an operation flow when blood pressure measurement is performed in a nighttime blood pressure measurement mode by the sphygmomanometer.

FIG. 7 includes sections (A), (B) and (C), which are hereinafter denoted as FIG. 7(A), FIG. 7(B), FIG. 7(C), respectively. FIG. 7(A) is a graph illustrating a time course of a cuff pressure PC associated with blood pressure measurement. FIG. 7(B) is a graph illustrating a time course of a pulse wave signal SM associated with the blood pressure measurement. FIG. 7(C) is a graph illustrating an envelope ENV set for a sequence of pulse wave amplitudes formed by the pulse wave signal SM.

FIG. 8 is a graph for explaining how to calculate a blood pressure in the nighttime blood pressure measurement mode.

FIGS. 9A and 9B are flowcharts illustrating how to determine whether or not a current blood pressure value measured in the nighttime blood pressure measurement mode is different from a past blood pressure value.

FIG. 10 is a flowchart illustrating a specific flow of processing of phenomenon discrimination and schedule resetting in the nighttime blood pressure measurement mode.

### DESCRIPTION OF EMBODIMENTS

Hereinafter, embodiments of the present invention will be described in detail with reference to the drawings.

(Configuration of Sphygmomanometer)

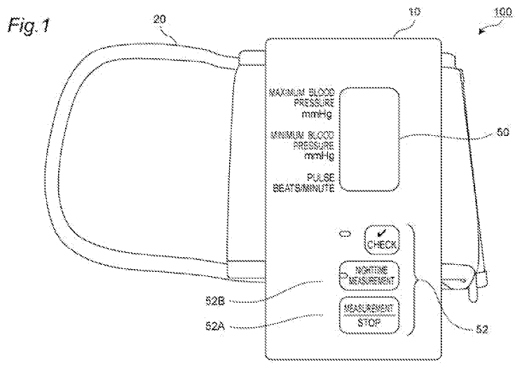

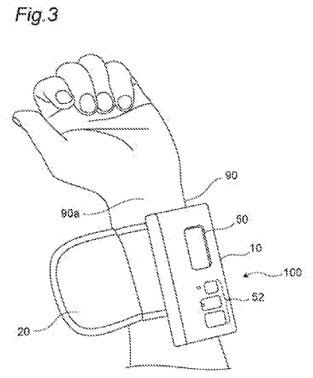

FIG. 1 illustrates an appearance of a wrist-type sphygmomanometer 100 according to an embodiment of the present invention. The sphygmomanometer 100 roughly includes a blood pressure measuring cuff 20 to be attached to a left wrist 90 (see FIG. 3 described later) as a measurement target site, and a main body 10 integrally attached to the cuff 20.

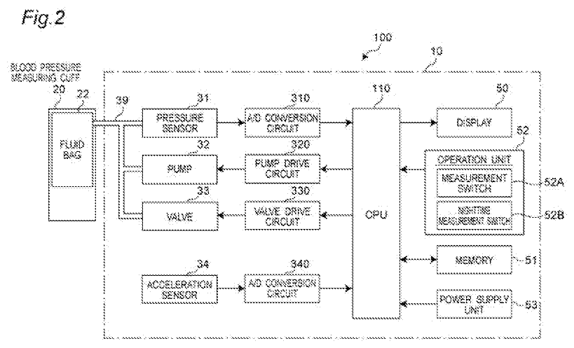

The cuff 20 is a general one for a wrist-type sphygmomanometer, and has an elongated band shape so as to surround the left wrist 90 along a circumferential direction. The cuff 20 contains a fluid bag 22 (see FIG. 2) for compressing the left wrist 90. Note that, in order to always maintain the cuff 20 in an annular shape, a curler having appropriate flexibility may be provided in the cuff 20.

As illustrated in FIG. 3, the main body 10 is integrally attached to a substantially central portion in a longitudinal direction of the belt-shaped cuff 20. In this example, the portion to which the main body 10 is attached is supposed to correspond to a palmar surface (surface on a palm side) 90a of the left wrist 90 in a worn state.

The main body 10 has a flat substantially rectangular parallelepiped shape along an outer peripheral surface of the cuff 20. The main body 10 is formed to be small and thin so as not to interfere with the sleep of a user (in this example, a subject; the same applies hereinafter). Furthermore, corner portions of the main body 10 are rounded (The corners are rounded.).

As illustrated in FIG. 1, a display 50 forming a display screen, and an operation unit 52 for inputting an instruction from the user are provided on a surface (top surface) on a side farthest from the left wrist 90 among outer surfaces of the main body 10.

In this example, the display 50 is a liquid crystal display (LCD), and displays predetermined information according to a control signal from a central processing unit (CPU) 110 described later. In this example, maximum blood pressure (unit; mmHg), minimum blood pressure (unit; mmHg), and pulse (unit; beats per minute) are displayed. Note that the display 50 may be an organic electro luminescence (EL) display, or may include a light emitting diode (LED).

The operation unit 52 inputs an operation signal corresponding to an instruction from the user to the CPU 110 described later. In this example, the operation unit 52 includes a measurement switch 52A for receiving a blood pressure measurement instruction from the user, and a nighttime measurement switch 52B for receiving an instruction to switch a mode between a normal blood pressure measurement mode and a nighttime blood pressure measurement mode. Here, the “normal blood pressure measurement mode” means a mode in which, when a blood pressure measurement instruction is input by the measurement switch 52A, blood pressure measurement is performed according to the blood pressure measurement instruction. The “nighttime blood pressure measurement mode” means a mode in which blood pressure measurement is automatically started according to a predetermined schedule so that the user can measure a blood pressure value during sleep. The predetermined schedule indicates, for example, a plan for measuring at regular time such as midnight 1:00, 2:00, or 3:00, a plan for measuring once every two hours after the nighttime measurement switch 52B is pressed, or the like.

Specifically, in this example, each of the measurement switch 52A and the nighttime measurement switch 52B is a momentary type (self-return type) switch, and is turned on only while being pushed down, and returns to an off state when being released.

When the measurement switch 52A is once pushed down while the sphygmomanometer 100 is in the normal blood pressure measurement mode, which means a blood pressure measurement instruction, the measurement target site (left wrist 90) is temporarily compressed by the cuff 20, and blood pressure measurement is executed by the oscillometric method. When the measurement switch 52A is pushed down again during the blood pressure measurement (for example, during pressurization of the cuff 20), it means an instruction to stop the blood pressure measurement, and the blood pressure measurement is immediately stopped.

When the nighttime measurement switch 52B is once pushed down while the sphygmomanometer 100 is in the normal blood pressure measurement mode, it means an instruction to transition to the nighttime blood pressure measurement mode, and the sphygmomanometer 100 transitions from the normal blood pressure measurement mode to the nighttime blood pressure measurement mode. In the nighttime blood pressure measurement mode, as described above, blood pressure measurement by the oscillometric method is automatically started according to the predetermined schedule. When the nighttime measurement switch 52B is pushed down again while the sphygmomanometer 100 is in the nighttime blood pressure measurement mode, it means an instruction to stop the nighttime blood pressure measurement mode, and the sphygmomanometer 100 transitions from the nighttime blood pressure measurement mode to the normal blood pressure measurement mode.

Even while the sphygmomanometer 100 is in the nighttime blood pressure measurement mode, the user may instruct blood pressure measurement by interrupting by pressing the measurement switch 52A separately from the predetermined schedule. At that time, in response to the interruption blood pressure measurement instruction, the measurement target site (left wrist 90) is temporarily compressed by the cuff 20, and blood pressure measurement is executed by the oscillometric method.

FIG. 2 illustrates a block configuration of the sphygmomanometer 100.

As described above, the cuff 20 includes the fluid bag 22 for compressing the left wrist 90 as the measurement target site. The fluid bag 22 and the main body 10 are connected by an air pipe 39 so as to be capable of fluid communication.

In addition to the display 50 and the operation unit 52 described above, the main body 10 is mounted with a CPU 110 as a control unit, a memory 51 as a storage unit, a power supply unit 53, an acceleration sensor 34, a pressure sensor 31, a pump 32, and a valve 33. Further, the main body 10 is mounted with an A/D conversion circuit 310 that converts an output of the pressure sensor 31 from an analog signal to a digital signal, a pump drive circuit 320 that drives the pump 32, a valve drive circuit 330 that drives the valve 33, and an A/D conversion circuit 340 that converts an output of the acceleration sensor 34 from an analog signal to a digital signal. The pressure sensor 31, the pump 32, and the valve 33 are commonly connected to the fluid bag 22 through the air pipe 39 so as to be capable of fluid communication.

The memory 51 stores a program for controlling the sphygmomanometer 100, data used for controlling the sphygmomanometer 100, setting data for setting various functions of the sphygmomanometer 100, data of a measurement result of a blood pressure value, a pulse rate, a pulse wave interval, output data of the acceleration sensor 34, and the like. In addition, the memory 51 is used as a work memory or the like when the program is executed.

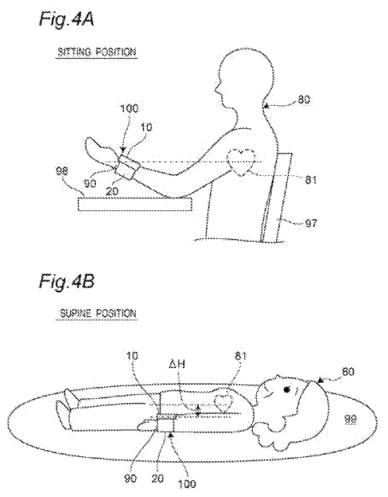

In particular, in this example, the memory 51 stores an algorithm for a sitting position and an algorithm for a supine position as algorithms for blood pressure calculation by the oscillometric method. Here, as illustrated in FIG. 4A, the “sitting position” means a posture in which the user 80 wearing the sphygmomanometer 100 on the left wrist 90 sits on a chair 97 or the like, and holds the left wrist 90 (and the sphygmomanometer 100) at a height level of a heart 81 by raising the left wrist 90 obliquely (hand is up, elbow is down) in front of a trunk with the left elbow attached to a table 98. This posture eliminates a height difference between the left wrist 90 and the heart 81 of the user 80, and thus is recommended to increase a blood pressure measurement accuracy. On the other hand, as illustrated in FIG. 4B, the “supine position” means a posture in which the user 80 wearing the sphygmomanometer 100 on the left wrist 90 lies on his/her back on a horizontal floor surface 99 or the like with the left elbow extended along the trunk. In this posture, a height difference ΔH between the left wrist 90 (and the sphygmomanometer 100) and the heart 81 of the user 80 occurs (the height of the heart 81 is higher than the height of the left wrist 90), so that the blood pressure measurement value deviates. In addition, since the left elbow is bent in the sitting position (FIG. 4A) while the left elbow is extended in the supine position (FIG. 4B), the blood pressure measurement value may be deviated due to bending and stretching of the left elbow. In order to eliminate such a deviation of the blood pressure measurement value in the supine position with respect to the blood pressure measurement value in the sitting position, it is preferable to change the blood pressure calculation algorithm in the case of measuring the blood pressure in the supine position with respect to the blood pressure calculation algorithm in the case of measuring the blood pressure in the sitting position. For this reason, in this example, the memory 51 stores the algorithm for the sitting position and the algorithm for the supine position as the algorithms for the blood pressure calculation by the oscillometric method. A specific method of calculating the blood pressure using these algorithms will be described later.

Further, in this example, as shown in a time difference table of the following Table 1, the memory 51 stores in advance a relative time difference for setting time of remeasurement for each of a plurality of predetermined types of phenomena that can occur in a subject in the nighttime blood pressure measurement mode. Here, in this example, the “plurality of predetermined types of phenomena” refers to four types of phenomena that can affect a blood pressure value, such as a change in a sleep state, occurrence of an irregular pulse wave, a change in a posture, and body motion. The “change in a sleep state” refers to a change in sleep depth, for example, a change from non-REM sleep (deep sleep) to REM sleep (light sleep), a change from REM sleep (light sleep) to an awake state, or the like. The “occurrence of an irregular pulse wave” refers to a state in which disturbance (including arrhythmia) occurs in a pulse wave that should be originally repeated at a constant cycle and constant intensity. The “change in a posture” refers to a phenomenon in which the subject shifts from a certain posture (for nighttime blood pressure measurements, typically a supine position) to another posture. The “body motion” refers to body motion (for example, repetitive motion) that does not correspond to a change in a posture. In this example, a time difference “30 minutes” is stored for the change in a posture, a time difference “5 minutes” is stored for the body motion, a time difference “15 minutes” is stored for the change in a sleep state, and a time difference “5 minutes” is stored for the occurrence of an irregular pulse wave. These time differences are empirically set in consideration of normal time during which a corresponding phenomenon continues. A specific method of setting the time of remeasurement using the time difference table will be described later.

TABLE 1
Time Difference Table

| Phenomena | Time Difference |
|---|---|
| Change in sleep state | 15 minutes |
| Occurrence of irregular pulse wave | 5 minutes |
| Change in posture | 30 minutes |
| Body motion | 5 minutes |

The CPU 110 illustrated in FIG. 2 controls the entire operation of the sphygmomanometer 100. Specifically, the CPU 110 acts as a pressure control unit according to a program for controlling the sphygmomanometer 100 stored in the memory 51, and performs control to drive the pump 32 and the valve 33 according to an operation signal from the operation unit 52. Furthermore, the CPU 110 acts as a blood pressure measurement unit, calculates a blood pressure value using an algorithm for blood pressure calculation by the oscillometric method, and controls the display 50 and the memory 51.

In this example, the power supply unit 53 includes a secondary battery, and supplies power to each unit of the CPU 110, the pressure sensor 31, the pump 32, the valve 33, the acceleration sensor 34, the display 50, the memory 51, the A/D conversion circuits 310 and 340, the pump drive circuit 320, and the valve drive circuit 330.

In this example, the acceleration sensor 34 includes a three-axis acceleration sensor integrally mounted on the main body 10, and outputs data indicating a direction (therefore, the posture of the subject wearing the main body 10) of a gravitational acceleration vector with respect to the main body 10, data indicating the body motion of the subject, and the like. The A/D conversion circuit 340 converts an output of the acceleration sensor 34 from an analog signal to a digital signal, and outputs the converted signal to the CPU 110. The acceleration sensor 34 serves as an element constituting a phenomenon discrimination unit to be described later, particularly, a posture determination unit and a body motion determination unit.

The pump 32 supplies air as a fluid to the fluid bag 22 through the air pipe 39 in order to pressurize a pressure (cuff pressure) in the fluid bag 22 included in the cuff 20. The valve 33 is opened and/or closed to discharge air in the fluid bag 22 through the air pipe 39 or to enclose air in the fluid bag 22 to control the cuff pressure. The pump drive circuit 320 drives the pump 32 based on a control signal given from the CPU 110. The valve drive circuit 330 opens and/or closes the valve 33 based on a control signal given from the CPU 110.

The pressure sensor 31 and the A/D conversion circuit 310 act as a pressure detection unit that detects the pressure of the cuff. The pressure sensor 31 is a piezoresistive pressure sensor in this example, and outputs the pressure (cuff pressure) in the fluid bag 22 contained in the cuff 20 through the air pipe 39 as an electric resistance due to the piezoresistive effect. The A/D conversion circuit 310 converts an output (electric resistance) of the pressure sensor 31 from an analog signal to a digital signal, and outputs the converted signal to the CPU 110. In this example, the CPU 110 acts as an oscillation circuit that oscillates at a frequency corresponding to the electric resistance from the pressure sensor 31, and acquires a signal indicating the cuff pressure according to the oscillation frequency. The pressure sensor 31 serves as an element constituting a blood pressure measurement unit, and also serves as an element constituting a phenomenon discrimination unit to be described later, particularly a sleep state determination unit and an irregular pulse wave determination unit.

(Blood Pressure Measurement Method)

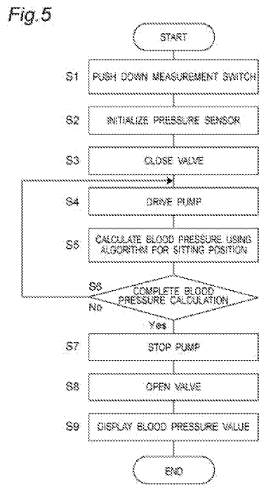

FIG. 5 illustrates an operation flow when the user performs blood pressure measurement in the normal blood pressure measurement mode by the sphygmomanometer 100. Note that, in this example, when the measurement switch 52A is continuously pressed for, for example, three seconds or more in a power-off state, a power source is turned on, and the normal blood pressure measurement mode is set by default.

As illustrated in FIG. 4A, it is assumed that the user 80 wearing the sphygmomanometer 100 on the left wrist 90 is in a sitting posture.

In this state, as shown in step S1 of FIG. 5, when the user pushes down the measurement switch 52A provided on the main body 10 to input a blood pressure measurement instruction, the CPU 110 initializes the pressure sensor 31 (step S2). Specifically, the CPU 110 initializes a processing memory area, and performs a 0 mmHg adjustment (The atmospheric pressure is set to 0 mmHg.) of the pressure sensor 31 in a state where the pump 32 is turned off (stopped) and the valve 33 is opened.

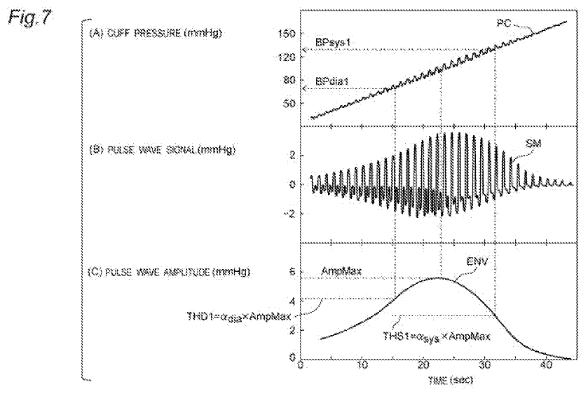

Next, the CPU 110 closes the valve 33 via the valve drive circuit 330 (step S3), and then turns on (starts) the pump 32 via the pump drive circuit 320 to start pressurization of the cuff 20 (fluid bag 22) (step S4). At this time, the CPU 110 controls a pressurization speed of a cuff pressure PC, which is a pressure in the fluid bag 22, as illustrated in FIG. 7(A) based on the output of the pressure sensor 31 while supplying air from the pump 32 to the fluid bag 22 through the air pipe 39.

Next, in step S5 of FIG. 5, the CPU 110 acts as a blood pressure measurement unit, and attempts to calculate a blood pressure value (maximum blood pressure (systolic blood pressure) and minimum blood pressure (diastolic blood pressure)) using the algorithm for the sitting position stored in the memory 51 on the basis of a pulse wave signal SM (fluctuation component due to a pulse wave included in the output of the pressure sensor 31) acquired at this time (see FIG. 7(B)).

At this time, when the blood pressure value cannot be calculated yet due to lack of data (No in step S6), the processing of steps S4 to S6 is repeated unless the cuffpressure PC reaches an upper limit pressure (For safety, for example, 300 mmHg is predetermined.).

Here, the CPU 110 calculates the blood pressure value as follows. That is, an envelope ENV as illustrated in FIG. 7(C) is set for a sequence of pulse wave amplitudes (peak-to-peak) formed by the pulse wave signal SM illustrated in FIG. 7(B) obtained from the cuff pressure PC when the cuff 20 is in the pressurization process. At the same time, two threshold levels THD1 and THS1 of ratios α_{dia} and α_{sys} predetermined for the sitting position are set with respect to a maximum value AmpMax of the envelope ENV. THD1 is a threshold level for a diastolic blood pressure, and is set as THD1=α_{dia}×AmpMax. Further, THS1 is a threshold level for a systolic blood pressure, and is set as THS1=α_{sys}×AmpMax. As an example, α_{dia}=0.75 and α_{sys}=0.4 (That is, THD1=0.75×AmpMax is set, and THS1=0.4×AmpMax is set.). Then, as illustrated in FIG. 7(A), the cuff pressures PC when the envelope ENV crosses the threshold levels THD1 and THS1 are calculated as the minimum blood pressure (diastolic blood pressure) BPdial and the maximum blood pressure (systolic blood pressure) BPsysl, respectively.

When the blood pressure value can be calculated in this manner (Yes in step S6), the CPU 110 turns off the pump 32 (step S7), opens the valve 33 (step S8), and performs control to exhaust the air in the cuff 20 (fluid bag 22).

Further, while repeating the processing of steps S4 to S6, the CPU 110 counts a pulse wave obtained from the cuff pressure PC, and calculates a pulse rate (unit; beats per minute).

Thereafter, the CPU 110 displays the calculated blood pressure value and pulse rate on the display 50 (step S9), and performs control to store data such as the blood pressure value and the pulse rate in the memory 51.

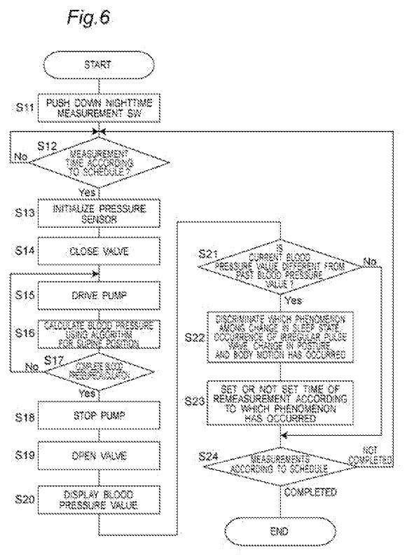

FIG. 6 illustrates an operation flow when the user performs blood pressure measurement in the nighttime blood pressure measurement mode by the sphygmomanometer 100. At the start of this flow, the sphygmomanometer 100 is assumed to be in the normal blood pressure measurement mode.

As shown in step S11 of FIG. 6, when the user pushes down the nighttime measurement switch 52B provided on the main body 10, the sphygmomanometer 100 transitions from the normal blood pressure measurement mode to the nighttime blood pressure measurement mode. In this example, in the nighttime blood pressure measurement mode, it is assumed that a schedule of measuring once every hour, for example, until 7:00 am after the nighttime measurement switch 52B is pressed is determined. Note that the present invention is not limited to this schedule, and a schedule for measuring on time such as 1:00 am, 2:00 am, and 3:00 am, for example, until 7:00 am from the time when the nighttime measurement switch 52B is pressed may be set.

Next, as shown in step S12 of FIG. 6, the CPU 110 determines whether or not it is measurement time specified in the schedule (of the nighttime blood pressure measurement mode). If it is not the measurement time specified in the schedule (No in step S12), the CPU waits for the measurement time specified in the schedule.

When the measurement time specified in the schedule is reached (Yes in step S12), the CPU 110 starts blood pressure measurement same as in steps S2 to S4 in FIG. 5 as shown in steps S13 to S15 in FIG. 6. That is, the CPU 110 first initializes the pressure sensor 31 (step S13).

Next, the CPU 110 closes the valve 33 via the valve drive circuit 330 (step S14), and then turns on (starts) the pump 32 via the pump drive circuit 320 to start pressurization of the cuff 20 (fluid bag 22) (step S15). At this time, the CPU 110 controls a pressurization speed of the cuff pressure PC in the same manner as illustrated in FIG. 7(A).

Next, in step S16 of FIG. 6, the CPU 110 acts as a blood pressure measurement unit, and attempts to calculate a blood pressure value (maximum blood pressure (systolic blood pressure) and minimum blood pressure (diastolic blood pressure)) using the algorithm for the supine position based on the pulse wave signal SM (fluctuation component due to the pulse wave included in the output of the pressure sensor 31) acquired at this time (similar to that illustrated in FIG. 7(B)).

At this time, when the blood pressure value cannot be calculated yet due to lack of data (No in step S17), the processing of steps S15 to S17 is repeated unless the cuff pressure PC reaches an upper limit pressure (For safety, for example, 300 mmHg is predetermined.).

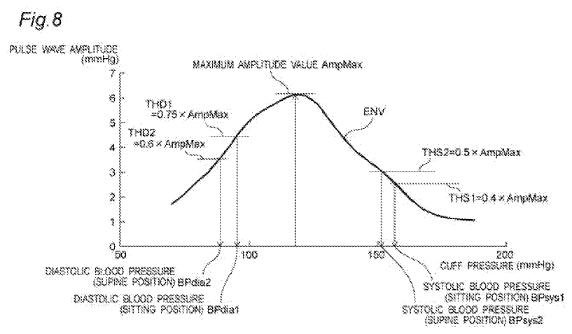

Here, the CPU 110 calculates the blood pressure value as follows. That is, the envelope ENV (similar to that illustrated in FIG. 7(C)) as illustrated in FIG. 8 is set for a sequence of the pulse wave amplitudes (peak-to-peak) formed by the pulse wave signal SM obtained from the cuff pressure PC when the cuff 20 is in the pressurization process. In the algorithm for the supine position, as illustrated in FIG. 8, as the threshold level for the diastolic blood pressure, THD2=0.6AmpMax is used instead of THD1=0.75×AmpMax, and as the threshold level for the systolic blood pressure, THS2=0.5×AmpMax is used instead of THS1=0.4×AmpMax. As a result, deviation of the blood pressure measurement value in the supine position (FIG. 4B) from the blood pressure measurement value in the sitting position (FIG. 4A) is eliminated. Then, the cuff pressures PC at the time when the envelope ENV crosses the currently set threshold levels THD2 (=0.6×AmpMax) and THS2 (=0.5×AmpMax) for the supine position are calculated as the minimum blood pressure (diastolic blood pressure) BPdia2 and the maximum blood pressure (systolic blood pressure) BPsys2, respectively.

In the nighttime blood pressure measurement mode, it is usually expected that the user is in a supine position. Therefore, by using the algorithm for the supine position, the blood pressure values (the maximum blood pressure and the minimum blood pressure) can be stably and accurately calculated.

When the blood pressure value (current blood pressure value) can be calculated in this manner (Yes in step S17), the CPU 110 turns off the pump 32 (step S18), opens the valve 33 (step S19), and performs control to exhaust the air in the cuff 20 (fluid bag 22).

Further, while repeating the processing of steps S15 to S17, the CPU 110 counts the pulse wave obtained from the cuff pressure PC and calculates a pulse rate (unit; beats per minute), and a pulse wave interval (unit; seconds) for the phenomenon discrimination described later, especially for sleep state determination and irregular pulse wave determination. At the same time, the CPU 110 acquires output data of the acceleration sensor 34 for posture determination and body motion determination described later.

Thereafter, the CPU 110 displays the calculated blood pressure value and pulse rate on the display 50 (step S20), and performs control to store data of the current blood pressure value, pulse rate, and pulse wave interval, and output data of the acceleration sensor 34 in the memory 51.

When one blood pressure measurement specified in the schedule is completed in this manner, in step S21, the CPU 110 acts as a difference determination unit to determine whether or not the current blood pressure value is different from the past blood pressure value. Specifically, this determination is made as follows. Note that, in this determination, it is assumed that the current blood pressure value and the past blood pressure value are compared between the maximum blood pressures (systolic blood pressures).

i) How to determine whether or not current blood pressure value is different from past blood pressure value Part 1

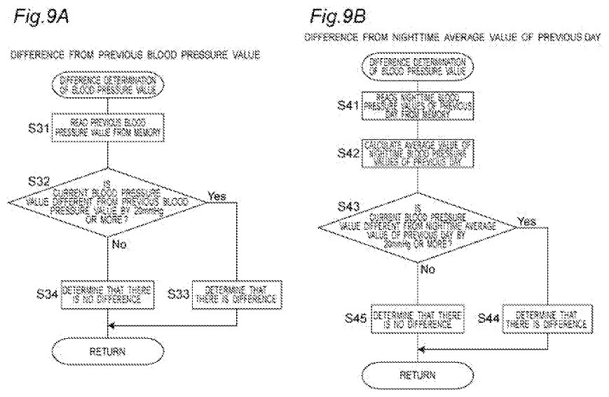

For example, it is assumed that a previous blood pressure value as the past blood pressure value is acquired at 2:00 am and the current blood pressure value is acquired at 3:00 am. In this case, as shown in step S31 of FIG. 9A, the CPU 110 reads the previous (in this example, 2:00 am) blood pressure value from the memory 51. Next, as shown in step S32, the CPU 110 determines whether or not the current (in this example, 3:00 am) blood pressure value differs from the previous blood pressure value by exceeding a predetermined allowable range (in this example, 20 mmHg). Here, when the current blood pressure value is different from the previous blood pressure value by 20 mmHg or more (Yes in step S32), it is determined that the current blood pressure value is “different” from the past blood pressure value (step S33). On the other hand, when a difference between the current blood pressure value and the previous blood pressure value is less than 20 mmHg (No in step S32), it is determined that the current blood pressure value is “not different” from the past blood pressure value (step S34).

ii) How to determine whether or not current blood pressure value is different from past blood pressure value Part 2

Further, it is assumed that, as the past blood pressure value, a plurality of nighttime blood pressure values are measured according to a schedule of the nighttime blood pressure measurement mode of the previous day, and stored in the memory 51. The current blood pressure value is assumed to be acquired at 3:00 am as in the above example. In this case, as shown in step S41 of FIG. 9B, the CPU 110 reads all the nighttime blood pressure values of the previous day from the memory 51. Next, as shown in step S42, the CPU 110 calculates an average value of the nighttime blood pressure values of the previous day. Next, as shown in step S43, the CPU 110 determines whether or not the current blood pressure value (in this example, 3:00 am) is different from the nighttime average value (average value of the nighttime blood pressure values) of the previous day by exceeding a predetermined allowable range (in this example, 20 mmHg). Here, when the current blood pressure value is different from the previous blood pressure value by 20 mmHg or more (Yes in step S43), it is determined that the current blood pressure value is “different” from the past blood pressure value (step S44). On the other hand, when a difference between the current blood pressure value and the previous blood pressure value is less than 20 mmHg (No in step S43), it is determined that the current blood pressure value is “not different” from the past blood pressure value (step S45).

Typically, the CPU 110 determines whether or not the current blood pressure value is different from the past blood pressure value by one of predetermined determination methods between a determination method in FIG. 9A and a determination method in FIG. 9B. However, the present invention is not limited thereto, and the CPU 110 may perform the determination by both FIGS. 9A and 9B each time the current measurement is performed, and may determine that the current blood pressure value is “different” from the past blood pressure value when it is determined that there is a “difference” in either one of the cases. As a result, when there is a possibility that the current measurement value includes a measurement error, this can be widely detected. Alternatively, the CPU 110 may determine that the current blood pressure value is “different” from the past blood pressure value only when “different” is determined according to both determination methods. As a result, only in a case where there is a high possibility that the current measurement value includes a measurement error, the processing of phenomenon discrimination and schedule resetting (steps S22 and S23) described later can be performed, and power saving can be measured.

In this manner, in step S21 of FIG. 6, it is determined whether or not the current blood pressure value is different from the past blood pressure value. As a result, it is determined whether or not there is a possibility that the current blood pressure value includes a measurement error.

Here, when it is determined that the current blood pressure value is “not different” from the past blood pressure value (No in step S21), the process proceeds to step S24, and the CPU 110 determines whether or not all the blood pressure measurements specified in the schedule are completed.

Here, as long as the blood pressure measurement is still scheduled according to the schedule (“not completed” in step S24), the process returns to step S12. Then, it waits for the next measurement time specified in the schedule (No in step S12). When the next measurement time specified in the schedule is reached (Yes in step S12), the CPU 110 repeats the processing in steps S13 to S20.

On the other hand, when it is determined in step S21 of FIG. 6 that the current blood pressure value is “different” from the past blood pressure value (Yes in step S21), the process proceeds to steps S22 and S23, and the CPU 110 acts as a phenomenon discrimination unit and a schedule resetting unit. That is, when the current blood pressure value is different from the past blood pressure value beyond the allowable range (in the above example, 20 mmHg), the phenomenon discrimination unit discriminates which phenomenon among a plurality of predetermined types of phenomena (in this example, four types of phenomena that can affect the blood pressure value, such as a change in a sleep state, occurrence of an irregular pulse wave, a change in a posture, and body motion) has occurred in the subject (step S22). Furthermore, as the schedule resetting unit, time of remeasurement with respect to the measurement time of the current blood pressure value is variably set according to which phenomenon among the plurality of types of phenomena has occurred (step S23).

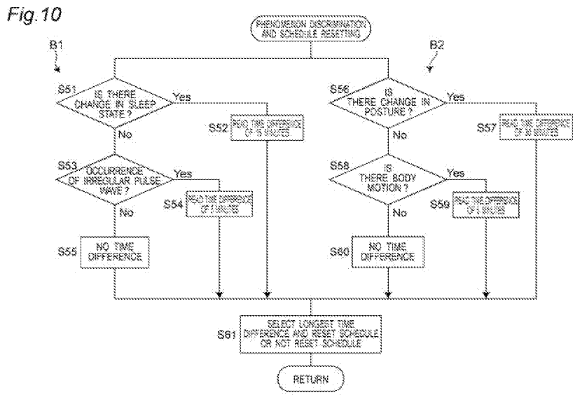

Specifically, processing of the phenomenon discrimination and the schedule resetting is performed according to a flow illustrated in FIG. 10. In this example, processing B1 in steps S51 to S55 and processing B2 in steps S56 to S60 in FIG. 10 are performed in parallel. The processing B1 includes a phenomenon discrimination process based on data of the pulse rate and the pulse wave interval calculated from the output of the pressure sensor 31. The processing B2 includes a phenomenon discrimination process based on data indicating the direction (therefore, the posture of the subject wearing the main body 10) of the gravitational acceleration vector with respect to the main body 10 and data indicating the body motion of the subject, which are obtained from the output of the acceleration sensor 34.

In the processing B1, first, in step S51, the CPU 110 acts as a sleep state determination unit, and determines whether or not a sleep state of the subject has changed based on the data of the pulse rate stored in the memory 51. Specifically, the CPU 110 detects whether or not the sleep state of the subject is a deep sleep state (non-REM sleep), a light sleep state (REM sleep), or an awake state from the sleep state from a change in the pulse rate by a known method disclosed in, for example, JP 2001-061819 A and JP 2007-199025 A. Here, as a simple example, when the pulse rate changes from the past average value (for example, 70 beats per minute) by more than a predetermined allowable range ±20%, the CPU 110 determines that the sleep state of the subject changes from the original non-REM sleep to the REM sleep or the awake state (Yes in step S51). At this time, the CPU 110 reads, as a candidate, a time difference of 15 minutes corresponding to the “change in a sleep state” from the time difference table (see Table 1) in the memory 51 (step S52). On the other hand, in other cases, the CPU 110 determines that there is no change in the sleep state (No in step S51), and proceeds to step S53.

In step S53, the CPU 110 acts as an irregular pulse wave determination unit, and determines whether or not an irregular pulse wave has occurred on the basis of the data of the pulse wave interval stored in the memory 51. Specifically, the CPU 110 determines that an irregular pulse wave has occurred when the pulse wave interval deviates by ±25% or more from the past average pulse wave interval by a known method disclosed in, for example, JP 2018-102670 A and JP 2019-115614 A (Yes in step S53). At this time, the CPU 110 reads, as a candidate, a time difference of 5 minutes corresponding to the “occurrence of an irregular pulse wave” from the time difference table (see Table 1) in the memory 51 (step S54). On the other hand, if not, the CPU 110 determines that the pulse wave is a regular pulse wave (No in step S53). At this time, the CPU 110 sets no time difference (0) as a candidate (step S55).

In the processing B2, first, in step S56, the CPU 110 acts as a posture determination unit, and determines whether or not a posture of the subject has changed on the basis of the output data of the acceleration sensor 34 stored in the memory 51, particularly, the data indicating the direction of the gravitational acceleration vector with respect to the main body 10. Specifically, the CPU 110 determines that the posture of the subject has changed in a case where the direction of the gravitational acceleration vector with respect to the main body 10 exceeds a predetermined threshold value by a known method disclosed in, for example, JP 3297971 B2 and JP 2013-183975 A (Yes in step S56). At this time, the CPU 110 reads, as a candidate, a time difference of 30 minutes corresponding to the “change in a posture” from the time difference table (see Table 1) in the memory 51 (step S57). On the other hand, if not, the CPU 110 determines that there is no change in a posture (No in step S56), and proceeds to step S58.

In step S58, the CPU 110 acts as a body motion determination unit, and determines whether or not there is body motion of the subject based on the output data of the acceleration sensor 34 stored in the memory 51, particularly, a change in the output of the acceleration sensor 34. Specifically, the CPU 110 determines whether or not there is body motion of the subject based on a change in the output of the acceleration sensor 34 by a known method disclosed in, for example, JP 2017-118982 A. That is, during the blood pressure measurement, average values <αx>, <αy>, and <αz>of the outputs αx, αy, and αz of the acceleration sensor 34 are obtained every unit period (for example, 1 second or several seconds), and variation amounts (αx−<αx>), (αy−<αy>), and (αz−<αz>) in which the acceleration outputs αx, αy, and αz at each time in the unit period vary with respect to the average values <αx>, <αy>, and <αz> are obtained. Then, when the square root sum of the variations {(αx−x>)²+(αy−<αy>)²+(αz−<αz>)²}^{1/2} exceeds a predetermined threshold (Δα.), it is determined that there is body motion (Yes in step S58). At this time, the CPU 110 reads a time difference of 5 minutes corresponding to the “body motion” as a candidate from the time difference table (see Table 1) of the memory 51 (step S59). On the other hand, if not, the CPU 110 determines that there is no body motion (No in step S58). At this time, the CPU 110 sets no time difference (0) as a candidate (step S60).

After the processing B1 and B2, in step S61, the CPU 110 acts as a schedule resetting unit, and sets the time of remeasurement by adding a relative time difference read from the time difference table to the measurement time of the current blood pressure value. For example, if the measurement time of the current blood pressure value is 3:00 am and the read relative time difference is only 15 minutes according to the “change in a sleep state”, the time of the remeasurement is set to 3:15 am. As a result, the time of the remeasurement can be smoothly set.

Here, when two or more phenomena among the plurality of types of phenomena occur in an overlapping manner at the measurement time of the current blood pressure value, two or more relative time differences are read from the time difference table by the processing B1 and B2. For example, when the change in a posture and the occurrence of the irregular pulse wave occur in an overlapping manner at the measurement time of the current blood pressure value, the relative time difference of 30 minutes according to the “change in a posture” and the relative time difference of 5 minutes according to the “occurrence of the irregular pulse wave” are read out by the processing B1 and B2. At this time, in step S61, the CPU 110 selects the longest time difference among the relative time differences read from the time difference table for the two or more phenomena that have occurred in an overlapping manner. In the above example, the longest time difference of 30 minutes is selected from the relative time difference of 30 minutes according to the “change in a posture” and the relative time difference of 5 minutes according to the “occurrence of an irregular pulse wave”. At this time, the CPU 110 adds the selected longest time difference of 30 minutes to the measurement time of the current blood pressure value (for example, it is set to 3:00 am.), and sets the remeasurement time to 3:30 am. In this manner, the CPU 110 sets the time of the remeasurement according to the phenomenon that may last the longest among the two or more phenomena that have occurred in an overlapping manner. As a result, it is possible to avoid a situation in which the remeasurement is started while a certain phenomenon (αphenomenon that continues for the longest time. In the above example, the change in posture) is still continuing among the two or more phenomena that have occurred in an overlapping manner.

Note that, if no time difference (0) is a candidate by the processing B1 and B2 (in particular, steps S55 and S60), the CPU 110 does not set the time for remeasurement in step S61.

When the process of the phenomenon discrimination and the schedule resetting (FIG. 10, that is, steps S22 and S23 in FIG. 6) is completed in this manner, the process proceeds to step S24 of FIG. 6, and it is determined whether or not all the blood pressure measurements specified in the schedule (This includes the remeasurement set in the processing of the phenomenon discrimination and the schedule resetting described above.) are completed.

Here, as long as the blood pressure measurement is still scheduled according to the schedule (“not completed” in step S24), the process returns to step S12. Then, it waits for the next measurement time specified in the schedule (No in step S12).

When the next measurement time specified in the schedule is reached (Yes in step S12), the CPU 110 repeats the processing in steps S13 to S20. In this way, as long as the blood pressure measurement is still scheduled according to the schedule (“not completed” in step S21), the CPU repeats the measurement, and when all the blood pressure measurements specified in the schedule are completed (“end” in step S24), the CPU 110 ends the nighttime blood pressure measurement mode.

As described above, according to the sphygmomanometer 100, when the current blood pressure value may include the measurement error (Yes in step S21 in FIG. 6), the time of remeasurement can be appropriately set according to the phenomenon that has occurred in the subject (steps S22 and S23 in FIG. 6). As a result, it is possible to avoid that the time of the remeasurement is too late for the phenomenon that has occurred or the time of the remeasurement is too early for the phenomenon that has occurred.

Furthermore, since the sphygmomanometer 100 is of a type that presses a wrist (In the above example, the left wrist 90 is used, but a right wrist may be used.) as a measurement target site, it is expected that the sphygmomanometer is less likely to disturb the sleep of the user (subject) than a type that presses an upper arm (Imai et al., “Development and evaluation of a home nocturnal blood pressure monitoring system using a wrist-cuff device”, Blood Pressure Monitoring 2018, 23, P318-326). Therefore, the sphygmomanometer 100 is suitable for the nighttime blood pressure measurement.

In addition, since the sphygmomanometer 100 is integrally and compactly configured as a wrist-type sphygmomanometer, handling by the user becomes convenient.

Furthermore, according to the sphygmomanometer 100, it is possible to determine whether or not four types of phenomena such as a change in a sleep state, occurrence of an irregular pulse wave, a change in a posture, and body motion have occurred as the plurality of types of phenomena using a relatively small number of hardware elements (In particular, the pressure sensor 31 and the acceleration sensor 34).

### Modified Example

Note that, in the above-described embodiment, the blood pressure is calculated in the pressurization process of the cuff 20 (fluid bag 22), but the present invention is not limited to this. The blood pressure may be calculated in the depressurization process of the cuff 20.

In the above embodiment, the blood pressure measurement instruction and the instruction to shift to the nighttime blood pressure measurement mode are input by the measurement switch 52A and the nighttime measurement switch 52B as the operation units provided in the main body 10, but the present invention is not limited thereto. For example, a communication unit capable of wireless communication may be mounted on the main body 10, and a blood pressure measurement instruction and a shift instruction to the nighttime blood pressure measurement mode may be input from a smartphone or the like existing outside the sphygmomanometer 100 via the communication unit.

Further, in the above embodiment, the main body 10 is provided integrally with the cuff 20, but the present invention is not limited to this. The main body 10 may be configured as a separate body from the cuff 20, and may be connected to the cuff 20 (fluid bag 22) through a flexible air tube so as to be able of fluid communication.

The above-described blood pressure measurement method (In particular, the operation flow of FIGS. 5, 6, 9, and 10) may be recorded as software (a computer program) on a non-transitory recording medium capable of storing data, such as a compact disc (CD), a digital universal disc (DVD), or a flash memory. By installing software recorded on such a recording medium in a substantial computer device such as a personal computer, a personal digital assistant (PDA), or a smartphone, the computer device can be caused to execute the above-described blood pressure measurement method.

In the above embodiment, the oscillometric method is adopted as the blood pressure measurement method, but the present invention is not limited thereto. As a blood pressure measurement method, a method of providing a microphone and observing a Korotkoff sound (Korotkoff method) may be adopted.

As described above, a sphygmomanometer of the present disclosure is a sphygmomanometer that performs blood pressure measurement by temporarily pressing a measurement target site of a subject with a blood pressure measuring cuff,

the sphygmomanometer having a nighttime blood pressure measurement mode in which the blood pressure measurement automatically starts according to a predetermined schedule, the sphygmomanometer comprising:

a storage unit that stores a measured blood pressure value;

a blood pressure measurement unit that automatically starts blood pressure measurement according to the schedule in the nighttime blood pressure measurement mode, and measures a blood pressure when the blood pressure measuring cuff is in a pressurization process or a depressurization process;

a difference determination unit that determines whether or not a current blood pressure value measured is different from a past blood pressure value stored in the storage unit beyond a predetermined allowable range;

a phenomenon discrimination unit that discriminates which phenomenon among a plurality of predetermined types of phenomena that can affect a blood pressure value has occurred in the subject when the current blood pressure value is different from the past blood pressure value beyond the allowable range; and

a schedule resetting unit that variably sets a time of remeasurement with respect to a measurement time of the current blood pressure value according to which phenomenon among the plurality of types of phenomena has occurred.

In the present specification, the current blood pressure value is “different from the past blood pressure value beyond a predetermined allowable range” typically means that the current blood pressure value is substantially different from the past blood pressure value in consideration of a measurement error. The “past blood pressure value” may be, for example, a previous blood pressure value obtained according to the schedule in the nighttime blood pressure measurement mode, or may be an average value of nighttime blood pressure values of the previous day obtained according to the schedule in the nighttime blood pressure measurement mode.

The “plurality of predetermined types of phenomena” typically refers to phenomena that can affect a blood pressure value, such as a change in a sleep state, occurrence of an irregular pulse wave, a change in a posture, and body motion. The “change in a sleep state” refers to a change in sleep depth, for example, a change from non-REM sleep (deep sleep) to REM sleep (light sleep), a change from REM sleep (light sleep) to an awake state, or the like. The “occurrence of an irregular pulse wave” refers to a state in which disturbance (including arrhythmia) occurs in a pulse wave that should be originally repeated at a constant cycle and constant intensity. The “change in a posture” refers to a phenomenon in which the subject shifts from a certain posture (for nighttime blood pressure measurements, typically a supine position) to another posture. The “body motion” refers to body motion (for example, repetitive motion) that does not correspond to a change in a posture.

“Whether or not a phenomenon has occurred” means whether or not a phenomenon has occurred at the measurement time of the blood pressure value. Note that a case where the plurality of predetermined types of phenomena have not occurred at all is also subject to the discrimination.

The “measurement time” of the blood pressure value refers to a time when the blood pressure measurement (usually required for about 1 minute to 2 minutes) is automatically started according to the schedule, and has the same meaning as a time when the blood pressure value is actually calculated in the pressurization process or the depressurization process of the blood pressure measuring cuff.

The sphygmomanometer of the present disclosure automatically starts blood pressure measurement according to the schedule in the nighttime blood pressure measurement mode. The blood pressure measurement unit measures a blood pressure when the blood pressure measuring cuff is in the pressurization process or the depressurization process (For example, the blood pressure value is calculated by the oscillometric method based on a pressure of the blood pressure measuring cuff.). The difference determination unit determines whether or not the measured current blood pressure value is different from the past blood pressure value stored in the storage unit beyond a predetermined allowable range. As a result, it is determined whether or not there is a possibility that the current blood pressure value includes a measurement error. When the current blood pressure value is different from the past blood pressure value beyond the allowable range, the phenomenon discrimination unit discriminates which phenomenon among a plurality of predetermined types of phenomena that can affect a blood pressure value has occurred in the subject. The schedule resetting unit variably sets the time of the remeasurement with respect to the measurement time of the current blood pressure value according to which phenomenon among the plurality of types of phenomena has occurred. Therefore, according to this sphygmomanometer, when the current blood pressure value may include a measurement error, the time of the remeasurement can be appropriately set according to the phenomenon that has occurred in the subject. As a result, it is possible to avoid that the time of the remeasurement is too late for the phenomenon that has occurred or the time of the remeasurement is too early for the phenomenon that has occurred.

In the sphygmomanometer according to one embodiment,

the storage unit includes a time difference table in which a relative time difference for setting the time of the remeasurement is stored in advance for each of the plurality of types of phenomena, and

the schedule resetting unit reads the relative time difference stored in the time difference table according to which phenomenon among the plurality of types of phenomena has occurred, and adds the read relative time difference to the measurement time of the current blood pressure value to set the time of the remeasurement.

Here, the “relative time difference for setting the time of the remeasurement” is empirically set in consideration of a normal time during which each corresponding phenomenon continues, for example, 30 minutes for the “change in a posture” and 5 minutes for the “body motion”.

In the sphygmomanometer according to the present embodiment, the time difference table stores in advance the relative time difference for setting the time of the remeasurement for each of the plurality of types of phenomena. The schedule resetting unit reads the relative time difference stored in the time difference table according to which phenomenon among the plurality of types of phenomena has occurred, and sets the time of the remeasurement by adding the read relative time difference to the measurement time of the current blood pressure value. As a result, the time of the remeasurement can be smoothly set.

In the sphygmomanometer according to one embodiment,

when two or more phenomena among the plurality of types of phenomena occur in an overlapping manner, the schedule resetting unit selects a longest time difference among relative time differences read from the time difference table for the two or more phenomena that have occurred in an overlapping manner.

In the sphygmomanometer according to the present embodiment, when two or more phenomena among the plurality of types of phenomena occur in an overlapping manner, the schedule resetting unit selects the longest time difference among relative time differences read from the time difference table for the two or more phenomena that have occurred in an overlapping manner. That is, the time of the remeasurement is set according to a phenomenon that may continue for the longest time among the two or more phenomena that have occurred in an overlapping manner. As a result, it is possible to avoid a situation in which the remeasurement is started while a certain phenomenon (a phenomenon that continues for the longest time) among the two or more phenomena that have occurred in an overlapping manner is still continuing.

The sphygmomanometer according to one embodiment further comprises a main body provided integrally with the blood pressure measuring cuff,

wherein the main body is equipped with the storage unit, the blood pressure measurement unit, the difference determination unit, the phenomenon discrimination unit, and the schedule resetting unit.

Here, the “blood pressure measurement unit” includes, for example, a pump that supplies a pressurizing fluid to the blood pressure measuring cuff, a valve that exhausts the fluid from the blood pressure measuring cuff, and elements that drive and control these pump, valve, and the like.

The sphygmomanometer of the present embodiment can be configured integrally and compactly. Therefore, handling by a user is convenient.

In the sphygmomanometer according to one embodiment,

the blood pressure measurement unit includes a pressure sensor that detects a pressure of the blood pressure measuring cuff, and acquires a blood pressure value by an oscillometric method based on the pressure of the blood pressure measuring cuff when the blood pressure measuring cuff is in a pressurization process or a depressurization process, and

the phenomenon discrimination unit includes:

a sleep state determination unit that determines whether or not a sleep state of the subject has changed based on a pulse rate obtained from the pressure of the blood pressure measuring cuff;

an irregular pulse wave determination unit that determines whether or not an irregular pulse wave has occurred based on an interval between pulse waves obtained from the pressure of the blood pressure measuring cuff;

a posture determination unit that includes an acceleration sensor integrally mounted on the main body, and determines whether or not a posture of the subject has changed based on an output of the acceleration sensor; and

a body motion determination unit that determines whether or not a body motion of the subject has occurred based on an output of the acceleration sensor.

In the sphygmomanometer according to the present embodiment, it is possible to determine whether or not four types of phenomena such as a change in a sleep state, occurrence of an irregular pulse wave, a change in a posture, and body motion have occurred as the plurality of types of phenomena using a relatively small number of hardware elements (in particular, a pressure sensor and an acceleration sensor).

In the sphygmomanometer according to one embodiment, the measurement target site is a wrist.

Since the sphygmomanometer of the present embodiment is a type that presses a wrist as the measurement target site, it is expected that the sphygmomanometer has less degree of disturbance of the sleep of the subject than a type that presses an upper arm (Imai et al., “Development and evaluation of a home nocturnal blood pressure monitoring system using a wrist-cuff device”, Blood Pressure Monitoring 2018, 23, P318-326). Therefore, this sphygmomanometer is suitable for the nighttime (sleep) blood pressure measurement.

In another aspect, a blood pressure measurement method according to the present disclosure is a blood pressure measurement method for a sphygmomanometer that performs blood pressure measurement by temporarily pressing a measurement target site of a subject with a blood pressure measuring cuff, the sphygmomanometer having a nighttime blood pressure measurement mode in which the blood pressure measurement is automatically started according to a predetermined schedule, and including a storage unit that stores a measured blood pressure value, the blood pressure measurement method comprising:

automatically starting blood pressure measurement according to the schedule in the nighttime blood pressure measurement mode, and measuring a blood pressure when the blood pressure measuring cuff is in a pressurization process or a depressurization process;

determining whether or not a current blood pressure value measured is different from a past blood pressure value stored in the storage unit beyond a predetermined allowable range;

discriminating which phenomenon among a plurality of predetermined types of phenomena that can affect a blood pressure value has occurred in the subject when the current blood pressure value is different from the past blood pressure value beyond the allowable range; and

variably setting a time of remeasurement with respect to a measurement time of the current blood pressure value according to which phenomenon among the plurality of types of phenomena has occurred.

According to the blood pressure measurement method of the present disclosure, when the current blood pressure value may include a measurement error, the time of the remeasurement can be appropriately set according to the phenomenon that has occurred in the subject. As a result, it is possible to avoid that the time of the remeasurement is too late for the phenomenon that has occurred or the time of the remeasurement is too early for the phenomenon that has occurred.

In yet another aspect, a computer-readable recording medium according to the present disclosure is a computer-readable recording medium non-transitorily storing a program for causing a computer to execute the above blood pressure measurement method.

By making a computer read the program stored in the computer-readable recording medium according to the present disclosure and causing a computer to execute the program, the blood pressure measurement method can be implemented.

As is clear from the above, according to the sphygmomanometer and the blood pressure measurement method of the present disclosure, when the current blood pressure value measured in the nighttime blood pressure measurement mode may include a measurement error, the time of the remeasurement can be appropriately set according to the phenomenon that has occurred in the subject. Further, according to the program stored in the computer-readable recording medium of the present disclosure, it is possible to cause a computer to execute such a blood pressure measurement method.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A sphygmomanometer that performs blood pressure measurement by temporarily pressing a measurement target site of a subject with a blood pressure measuring cuff,
the sphygmomanometer having a nighttime blood pressure measurement mode in which the blood pressure measurement automatically starts according to a predetermined schedule, the sphygmomanometer comprising:
a storage unit that stores a measured blood pressure value;
a blood pressure measurement unit that automatically starts blood pressure measurement according to the schedule in the nighttime blood pressure measurement mode, and measures a blood pressure when the blood pressure measuring cuff is in a pressurization process or a depressurization process;
a difference determination unit that determines whether or not a current blood pressure value measured is different from a past blood pressure value stored in the storage unit beyond a predetermined allowable range;
a phenomenon discrimination unit that discriminates which phenomenon among a plurality of predetermined types of phenomena that can affect a blood pressure value has occurred in the subject when the current blood pressure value is different from the past blood pressure value beyond the allowable range; and
a schedule resetting unit that variably sets a time of remeasurement with respect to a measurement time of the current blood pressure value according to which phenomenon among the plurality of types of phenomena has occurred.

2. The sphygmomanometer according to claim 1, wherein
the storage unit includes a time difference table in which a relative time difference for setting the time of the remeasurement is stored in advance for each of the plurality of types of phenomena, and
the schedule resetting unit reads the relative time difference stored in the time difference table according to which phenomenon among the plurality of types of phenomena has occurred, and adds the read relative time difference to the measurement time of the current blood pressure value to set the time of the remeasurement.

3. The sphygmomanometer according to claim 2, wherein
when two or more phenomena among the plurality of types of phenomena occur in an overlapping manner, the schedule resetting unit selects a longest time difference among relative time differences read from the time difference table for the two or more phenomena that have occurred in an overlapping manner.

4. The sphygmomanometer according to claim 3, comprising
a main body provided integrally with the blood pressure measuring cuff,
wherein the main body is equipped with the storage unit, the blood pressure measurement unit, the difference determination unit, the phenomenon discrimination unit, and the schedule resetting unit.

5. The sphygmomanometer according to claim 4, wherein
the blood pressure measurement unit includes a pressure sensor that detects a pressure of the blood pressure measuring cuff, and acquires a blood pressure value by an oscillometric method based on the pressure of the blood pressure measuring cuff when the blood pressure measuring cuff is in a pressurization process or a depressurization process, and
the phenomenon discrimination unit includes:
a sleep state determination unit that determines whether or not a sleep state of the subject has changed based on a pulse rate obtained from the pressure of the blood pressure measuring cuff;
an irregular pulse wave determination unit that determines whether or not an irregular pulse wave has occurred based on an interval between pulse waves obtained from the pressure of the blood pressure measuring cuff;
a posture determination unit that includes an acceleration sensor integrally mounted on the main body, and determines whether or not a posture of the subject has changed based on an output of the acceleration sensor; and
a body motion determination unit that determines whether or not a body motion of the subject has occurred based on an output of the acceleration sensor.

6. The sphygmomanometer according to claim 1, wherein
the measurement target site is a wrist.

7. A blood pressure measurement method for a sphygmomanometer that performs blood pressure measurement by temporarily pressing a measurement target site of a subject with a blood pressure measuring cuff, the sphygmomanometer having a nighttime blood pressure measurement mode in which the blood pressure measurement is automatically started according to a predetermined schedule, and including a storage unit that stores a measured blood pressure value, the blood pressure measurement method comprising:
automatically starting blood pressure measurement according to the schedule in the nighttime blood pressure measurement mode, and measuring a blood pressure when the blood pressure measuring cuff is in a pressurization process or a depressurization process;
determining whether or not a current blood pressure value measured is different from a past blood pressure value stored in the storage unit beyond a predetermined allowable range;
discriminating which phenomenon among a plurality of predetermined types of phenomena that can affect a blood pressure value has occurred in the subject when the current blood pressure value is different from the past blood pressure value beyond the allowable range; and
variably setting a time of remeasurement with respect to a measurement time of the current blood pressure value according to which phenomenon among the plurality of types of phenomena has occurred.

8. A computer-readable recording medium non-transitorily storing a program for causing a computer to execute the blood pressure measurement method according to claim 7.
